(19) United States
(12) Patent Application Publication
(10) Pub. No.: US 2002/0103403 A1
(43) Pub. Date: Aug. 1, 2002

(54) Process for preparing 3-alkoxypropanol

(76) Inventors: Torsten Hahm, Grundau-Rothenbergen (DE); Christian Ronge, Buchs im Aargau (DE); Manuela Kuhnen, Bekond (DE); Rudolf Vanheertum, Brasschaat (BE); Wolfgang Woll, Mainal-Dornigheim (DE)

Correspondence Address: PILLSBURY WINTHROP, LLP, P.O. BOX 10500, MCLEAN, VA, 22102, US

(21) Appl. No.: 09/973,954

(22) Filed: Oct. 11, 2001

Related U.S. Application Data
(60) Provisional application No. 60/247,968, filed on Nov. 14, 2000.

(30) Foreign Application Priority Data
Oct. 14, 2000 (EP) EP 00 122 489.8

(51) Int. Cl. C07C 41/18

(52) U.S. Cl. 568/678

(57) ABSTRACT

A process for preparing 3-alkoxypropanol by heterogenously catalysed hydrogenation of 3-alkoxypropionaldehyde in alcoholic solution, wherein at least one metal from the group iron, cobalt, nickel, copper, silver, molybdenum, tungsten, vanadium, chromium, rhodium, palladium, osmium, iridium, ruthenium and/or platinum on an oxidic support is used as catalyst.

The invention provides a process for preparing 3-alkoxypropanol by hydrogenating 3-alkoxypropionaldehyde.

Various processes for preparing 3-alkoxypropanol which start either with a C₂ and a C₁ unit or with a C₃ unit such as, for example, acrolein are known.

Thus, it is known that acrolein can first be alkoxylated in the presence of a catalyst, wherein 3-alkoxypropionaldehyde is formed. The reaction mixture formed during alkoxylation contains, after the removal of unreacted acrolein and without taking into account the solvent, in addition to about 95% 3-alkoxypropionaldehyde, other organic components in small amounts by weight. To prepare 3-alkoxypropanol, this reaction mixture is hydrogenated in the presence of hydrogenation catalysts.

The catalyst may be present in the suspended form per se or it may be a constituent of fixed bed catalysts. Homogeneous catalysts may also be used. Known suspension catalysts are Raney nickel, which may be doped with various other catalytically active metals, and platinum on activated carbon (DE 39 26 136).

The known process for catalytic hydrogenation has the disadvantage that the catalytically active element is discharged in the product stream in small amounts in the form of soluble compounds and thus additional working steps are required in order to remove the impurities thereby produced. This is observed in particular in the case of suspension catalysts such as, for example, Raney nickel.

In the case of nickel fixed bed heterogeneous catalysts, there is the additional disadvantage that the product is contaminated by nickel compounds.

Hydrogenation processes can be characterised by the conversions, selectivities and space-time yields which can be produced using them.

The conversion states how many moles of the reactant (here 3-alkoxypropionaldehyde) are converted into other substances as a result of hydrogenation. The information is usually given as a percentage of the moles of reactant initially introduced.

The selectivity of a hydrogenation process, on the other hand, is a measure of how many moles of the reactant are converted into the required product. For continuous hydrogenation processes, the space-time yield is another important characteristic and this cites the amount of product which can be produced per unit of time and per reaction volume.

During the large-scale industrial hydrogenation of 3-alkoxypropionaldehyde to give 3-alkoxypropanol, it is critical for the economic viability of the hydrogenation process and for the quality of the product that the conversion and the selectivity are as close as possible to 100%. Although, after hydrogenation, the 3-alkoxypropanol is separated from the alcohol and residual alkoxypropionaldehyde and also secondary products present in the product stream by distillation, this distillation procedure is made substantially more difficult by the presence of residual alkoxypropionaldehyde and secondary products. The lower are the conversion and selectivity, the poorer is the product quality which can be achieved.

In order to be able to prepare 3-alkoxypropanol economically, it is also important that the catalyst for hydrogenation of the 3-alkoxypropanal exhibits a high activity.

An object of the invention is to find a process in which the smallest possible amount of catalyst is required for preparing the 3-alkoxypropanol. It is intended, by using a small volume of catalyst, that the highest possible conversion of 3-alkoxypropanal to 3-alkoxypropanol be produced.

In hydrogenation processes, the conversion, selectivity and space-time yields are affected by the properties of the catalyst and by the hydrogenation conditions such as reaction temperature, hydrogen pressure, duration of hydrogenation or, in the case of continuous hydrogenation, by the space velocity LHSV (Liquid Hourly Space Velocity).

The Engelhard brochure “Exceptional Technologies” 1991 discloses that aliphatic carbonyl compounds can be hydrogenated to give the corresponding alcohols in the presence of ruthenium on aluminium oxide (Escalit).

The Degussa brochure “Powder Precious Metal Catalyst” (published June 1995) discloses that aliphatic aldehydes can be hydrogenated to give alcohols in the presence of ruthenium supported catalysts. Aluminium oxide is cited as a support.

A substantial quality criterion for the catalysts used in hydrogenation processes is the operational lifetime. Effective catalysts should ensure constant conversions and selectivities for the hydrogenation of 3-alkoxypropionaldehyde to give 3-alkoxypropanol during the course of the operational lifetime. Here, known hydrogenation processes in accordance with the prior art, in particular those based on nickel catalysts, exhibit unsatisfactory long-term stabilities. This requires frequent replacement of the entire catalyst packing, involving known problems associated with the waste-disposal and working-up of nickel-containing compounds.

Therefore, another object of the present invention is to develop a hydrogenation process which does not have the disadvantages of the processes according to the prior art.

The invention provides a process for preparing 3-alkoxypropanol, which is characterised in that during the heterogeneously catalysed hydrogenation of 3-alkoxypropionaldehyde in alcoholic solution at a temperature of 30 to 120° C., a hydrogen pressure of 5 to 300 bar and a pH 2.5 to 7.0, a supported catalyst which consists of an oxidic phase, preferably an oxidic phase which is stable in acid media, and on which at least one metal from the group iron, cobalt, nickel, copper, silver, molybdenum, tungsten, vanadium, chromium, rhodium, palladium, osmium, iridium, ruthenium and/or platinum, in particular ruthenium, platinum and/or nickel, is present in finely divided form, in an amount of 0.1 to 5 wt. % with respect to the oxidic phase, is used as catalyst.

In a preferred embodiment of the invention, the temperature may be 40 to 100° C., in particular 50 to 80° C. The pressure may preferably be 10 to 150, in particular 40 to 100 bar. The pH may preferably be 3 to 6, in particular 4 to 5.

As oxidic phases, substances from the group titanium dioxide, SiO₂, Al₂O₃ and/or their mixed oxides and also aluminium silicates, MgO, zeolites or zirconium dioxide, may be used.

These types of substances are described, for example, in Catalyst Supports and Supported Catalysts by Alvin B, Stiles Verlag, Butterworths 1987, chapters 2 and 3.

An oxidic phase which is stable in acid media is preferably used. These types of oxidic phases may be substances from the group titanium dioxide, SiO₂ and/or their mixed oxides and also aluminium silicates, MgO, zeolites or zirconium dioxide. Aluminium oxide is less resistant to acids.

In a preferred embodiment of the invention, oxides and mixed oxides of aluminium and in particular titanium and/or silicon can be used as oxidic phases.

A pyrogenically prepared titanium dioxide, in particular one prepared by flame hydrolysis, may be used as titanium dioxide.

The titanium dioxide used may be, for example, a so-called pyrogenic titanium dioxide obtained by flame hydrolysis from titanium tetrachloride, with a BET surface area of 40 to 60 m²/g and a total pore volume 0.25 to 0.75 ml/g, which has an average primary particle size of 20 nm, a density 3.7 g/cm³ and an X-ray structure consisting of 20 to 40% rutile and 80 to 60% anatase and in which impurities of silicon dioxide, aluminium oxide and iron oxide amount to less than 0.5 wt. %. Pyrogenic titanium oxide such as titanium dioxide P 25 from Degussa-Huls is particularly suitable as a support for the catalytically active components. It has a high BET specific surface area of on average 50 m²/g (measured in accordance with DIN 66131).

The oxides may be processed to give moulded articles such as, for example, pellets, granules or extrudates.

The oxidic phase may be coated using the Incipient Wetness Method, published in “Preparation of Catalyst”, Delmon, B., Jacobs, P. A., Poncald, G. (eds.), Amsterdam Elsevier, 1976, page 13.

For this purpose, the water take-up capacity of the support is determined. Then an aqueous ruthenium chloride solution with a concentration in accordance with the subsequent ruthenium coating is made up. The support is loaded with aqueous ruthenium chloride in accordance with its water take-up capacity. Then the loaded support is dried, preferably at 20 to 100° C. at atmospheric pressure under an inert gas atmosphere such as nitrogen, neon, helium, argon or air, reduced with hydrogen, preferably at a temperature of 100 to 500° C. for a time of 20 min to 24 hrs, with a hydrogen concentration of 1 to 100% mixed with nitrogen and optionally washed chloride-free, preferably to <100 ppm Cl⁻.

The alkoxy group in the alkoxypropanal may be a methoxy, ethoxy, propoxy, isopropoxy, butoxy, isobutoxy, pentoxy or isopentoxy group, etc.

In another embodiment of the invention, post-hydrogenation may be performed. This post-hydrogenation may be performed at a temperature of 100 to 180° C., a pressure of 5 to 300 bar and a pH of 2.5 to 7.0.

Post-hydrogenation may be performed with the same catalyst. However, the use of activated carbon as a catalyst support is also possible.

Post-hydrogenation has the advantage that the amount of other carbonyl compounds, which are difficult to remove by distillation alone, is reduced. Furthermore, acetates such as acrolein dialkyl acetate, or trialkoxypropane which are also formed, are reduced in amount.

Post-hydrogenation may advantageously be performed after 50 to 95% of the 3-alkoxypropanal has been converted.

The process according to the invention has the advantage that a higher conversion can be produced due to the higher activity of the catalyst.

Furthermore, the catalysts used have relatively long operating lifetimes.

### EXAMPLES

The catalysts are tested under steady-state conditions in order to be able to make statements about long-term behaviour. Hydrogenation is performed continuously in a trickle-bed unit with a reactor volume of 140 ml. The unit consists of a liquid storage tank, the liquid reactor and a liquid separator. The reaction temperature is adjusted via a heat-transfer medium/oil circuit. The pressure and hydrogen stream are controlled electronically. The aqueous alkoxypropionaldehyde solution is metered into the hydrogen stream using a pump and the mixture is supplied to the head of the reactor (trickle bed mode of operation). After passage through the reactor, the product formed is withdrawn from the separator at regular intervals. The concentration of alkoxypropionaldehyde in the reactant solution in all cases is 10 wt. %, the temperature is 40° C., the pressure is 40 bar, the liquid throughput LHSV is 1 h⁻¹.

Method used to prepare the catalysts:

1. The water take-up of the support, in g H₂O per 100 g of support, is determined.

2. To load 250 ml of support, 11.8 g of RuCl₃ are dissolved in 145 g of distilled water.

3. 250 ml of support are initially introduced into the coating pan and the RuCl₃ solution is poured over this while the pan is rotating.

4. The coated support is dried for 16 h in air and then heated to 200° C. in a tubular furnace.

5. The catalyst is reduced with hydrogen at 200° C. for 8 h.

6. The reduced catalyst is washed chloride-free, three times, using 40 ml of distilled water each time.

The supports used are characterised as follows:

| | |
|---|---|
| Support 1: | Silica gel from Grace (0.8-1.2 mm) |
| Name: | V432 |
| Support 2: | Activated carbon from Norit (diameter 2.3 mm) |
| Name: | Norit CNR 115 (olive stones) |
| Support 3: | Activated carbon from Norit (diameter 0.8 mm) |
| Name: | Norit ROX (peat carbon) |
| Support 4: | Titanium dioxide P25 (pyrogenic, prepared |
| | using flame hydrolysis, from Degussa-Hüls |
| | AG) |
| Support 5: | Al₂O₃ from Rhône-Poulenc |
| | (diameter 1.1-1.3 mm) |
| Name: | Spheralite 521 |

The following conditions were used when coating the supports:

| | | Water take-up | Support | RuCl₃ | Water |
|---|---|---|---|---|---|
| | Support | [g/100 g support] | [g] | [g] | [g] |
| Support 1 | SiO₂ V432 | 126 | 115 | 11.8 | 145 |
| Support 2 | Norit 1 Extra | 67 | 105 | 10.8 | 56 |
| Support 3 | Norit ROX 0.8 | 80 | 107 | 11.1 | 68 |
| Support 4 | TiO₂ EP0 535 565 | 25 | 100 | 10.2 | 14 |
| Support 5 | Al₂O₃ | 67 | 101 | 10.3 | 57 |

In contrast to known catalysts, the ruthenium catalysts on oxidic supports according to the invention exhibit no deactivation at all. The conversion remains constant. In particular, catalysts with ruthenium on SiO₂ and TiO₂ exhibit a very high activity.

1. A process for preparing 3-alkoxypropanol, wherein during the heterogeneously catalysed hydrogenation of 3-alkoxypropionaldehyde in alcoholic solution at a temperature of 30 to 120° C., a hydrogen pressure of 5 to 300 bar and a pH 2.5 to 7.0, a supported catalyst which consists of an oxidic phase and in which at least one metal from the group iron, cobalt, nickel, copper, silver, molybdenum, tungsten, vanadium, chromium, rhodium, palladium, osmium, iridium, ruthenium and/or platinum is present in finely divided form on the oxidic phase, in an amount of 0.1 to 5 wt. % with respect to the oxidic phase, is used as catalyst.

2. A process as claimed in claim 1, wherein a substance from the group titanium dioxide, SiO₂, Al₂O₃ and/or mixed oxides of these and also aluminium silicates, MgO, zeolites or zirconium oxide is used as the oxidic phase.

3. A process as claimed in claim 1, wherein a post-hydrogenation procedure is performed.
